(19) United States
(12) Patent Application Publication
(10) Pub. No.: US 2026/0347350 A1
(43) Pub. Date: Nov. 5, 2026

(54) PROCESS FOR THE PREPARATION OF ISAVUCONAZONIUM MONOSULFATE

(71) Applicant: ICROM S.P.A., Concorezzo (MB) (IT)

(72) Inventors: Marco Cipriano, Concorezzo (MB) (IT); Daniele De Zani, Concorezzo (MB) (IT); Gianpiero Mastronardo, Concorezzo (MB) (IT)

(73) Assignee: ICROM S.P.A., Concorezzo (MB) (IT)

(21) Appl. No.: 19/099,163

(22) PCT Filed: Jul. 27, 2023
(86) PCT No.: PCT/IB2023/057637

§ 371 Date: Jan. 28, 2025

(30) Foreign Application Priority Data
Aug. 1, 2022 (IT) 102022000016335

(51) Int. Cl. C07D 417/14

(52) U.S. Cl. CPC C07D 417/14

(57) ABSTRACT

The present invention concerns a process for the preparation of isavuconazonium monosulfate, the process allowing to obtain isavuconazonium monosulfate as a isolated powder in good yield, with high HPLC purity (>95%) and the proper amount of sulphate content, without involving the use of ionic resin, column chromatography or freeze-5 drying step.

### FIELD OF THE INVENTION

The present invention concerns a process for the preparation of isavuconazonium monosulfate, the process allowing to obtain isavuconazonium monosulfate as a isolated powder in good yield, with high HPLC purity (>95%) and the proper amount of sulphate content, without involving the use of ionic resin, column chromatography or freeze-drying step.

### State of the Art

Isavuconazonium (mono) sulfate is a systemic antifungal medication of the triazole class which is used to treat invasive aspergillosis and mucormycosis.

Isavuconazonium comprises an N-(3-acetoxypropyl)-N-methylamino-carboxymethyl group linked through an ester moiety to the triazole nitrogen in isavuconazole, as shown in the following formula (I):

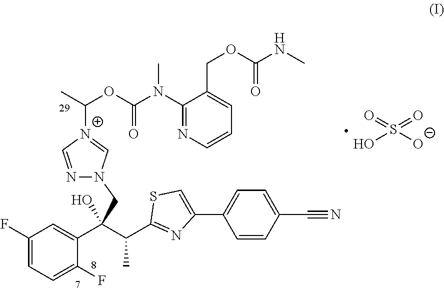 [SMILES: CNC(=O)OCc1cccnc1N(C)C(=O)OC(C)[n+]1cnn(C[C@](O)(c2cc(F)ccc2F)[C@@H](C)c2nc(-c3ccc(C#N)cc3)cs2)c1 ; O=S(=O)([O-])O]

The chemical name of the active substance isavuconazonium sulfate is 1-{(2R,3R)-3-[4-(4-cyanophenyl)-1,3-thiazol-2-yl]-2-(2,5-difluoro-phenyl)-2-hydroxybutyl}-4-[(IRS)-1-({methyl[3-({[(methylamino) acetyl]oxy}methyl)pyridin-2-yl]carbamoyl}oxy)ethyl]-1H-1,2,4-triazol-4-ium monosulfate (IUPAC), corresponding to the molecular formula C₃₅H₃₅F₂N₈O₅S·HSO₄ and has a relative molecular mass of 814.84 g/mol. The relative molecular mass of isavuconazole is 437.47. Isavuconazonium is indeed a prodrug of isavuconazole.

This compound is described by Procedure n. EMEA/H/C/002734/0000 (Cresemba-epar-public-assessmentreport_en2015) as a white, amorphous, hygroscopic powder. It is very soluble in water and over the pH range 1-7. It is also very soluble in methanol and sparingly soluble in ethanol. Two pKa values have been found and calculated to be 2.0 and 7.3. ISV sulfate in commercial products available on the market is reported to feature a purity of about 93%, however it was not possible to directly test whether this information is correct, as the starting ISV sulfate used in said commercial products is not available as such.

Isavuconazonium sulfate has three chiral centres. The stereochemistry of the active substance is introduced by one of the starting materials which is controlled by appropriate specification. The two centres, C7 and C8 in the isavuconazole moiety and in an intermediate of the active substance, have R configuration. The third chiral centre, C29, is not located on isavuconazole moiety and has both the R and S configurations. The non-defined stereo centre at C29 has been found in all batches produced so far to be racemic, according to said Procedure report. Erosion of stereochemical purity has not been observed in the current process. The active substance is a mixture of two epimers of C29. An enantiomer of drug substance was identified as C7 (S), C8 (S) and C29 (R/S) structure. The control of the stereochemistry of isavuconazonium sulfate is performed by chiral HPLC on the active substance and its two precursors. Subsequent intermediates are also controlled by relevant specification in the corresponding steps. Two crystal forms have been observed by recrystallisation studies. However, the manufacturing process as described yields amorphous form only.

Isavuconazonium sulfate is mentioned among the compounds disclosed in U.S. Pat. No. 7,459,561, however no description of the procedure to obtain the sulfate salt is described therein. Recently, other patent documents have addressed possible pathways of preparation of isavuconazonium sulfate, mainly involving ion different exchange steps and purification strategies.

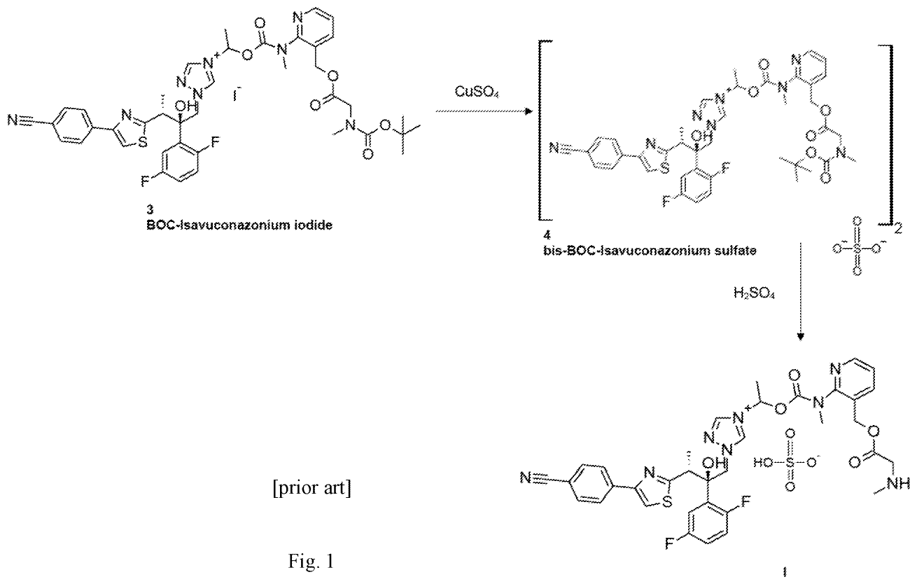

In particular, it is known from CN106916152A a preparation process starting from isavuconazonium as iodide hydrochloride salt, then converted into the corresponding free-base by using NaHCO₃ and then treated with H₂O₂ and sulfuric acid to obtain the oxidation of iodine into iodide in the presence of a source of sulfate anion (FIG. 1). However, this process gives no satisfactory results and a strong degradation of the molecules take places after treatment with hydrogen peroxide, thus demonstrating that this pathways is not suitable to produce the desired sulfate salt in high yield and HPLC purity. Moreover, the isolation of the salts requires lyophilisation. Lyophilisation is a technique that it energetically demanding and not very suitable for high scale production. The processes disclosed in the prior art also comprise chromatographic purifications and ion exchange chromatography for ion exchanges. Both these techniques are not suitable for higher scale production.

Therefore, there is a need for alternative process that does not comprise chromatographic purification, ion exchange chromatography and lyophilisation and that provides salts of compound of formula (I) in sufficient purity and yield.

Accordingly, it is an object of the present invention to provide a process of preparation of isavuconazonium monosulfate (or shortly “ISV SO₄²⁻”), which allows to obtain the desired salt in an appreciable high yield and purity, as well as can be performed in a convenient and expedite way.

### SUMMARY OF THE INVENTION

The above object has been achieved by a process of preparation comprising the steps of alkylation, ion exchange and desalification, as reported in claim 1.

The characteristics and the advantages of the present invention will become clear from the following detailed description, the working examples provided for illustrative purposes, and the annexes figures, wherein:

FIG. 1 shows the preparation pathway disclosed in CN106916152A, as a comparative prior art;

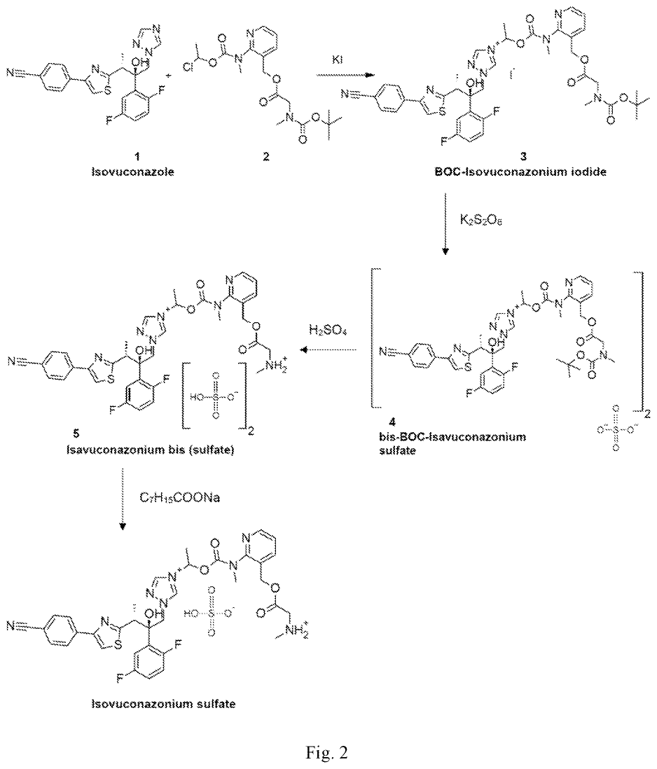

FIG. 2 shows a first preferred preparation pathway according to the present invention; and

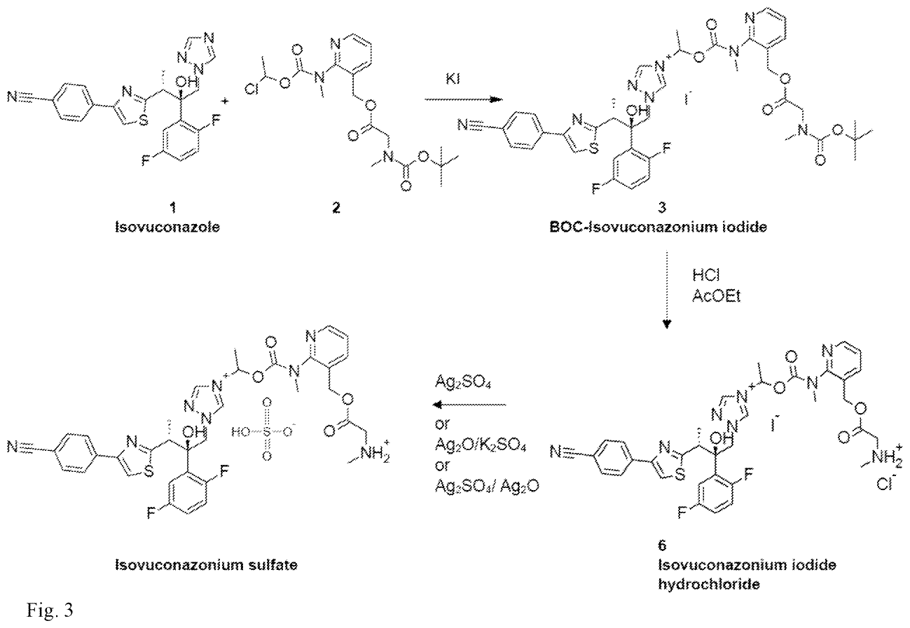

FIG. 3 shows a second preferred preparation pathway according to the present invention.

### DETAILED DESCRIPTION OF THE INVENTION

The present invention therefore relates to a process for the preparation of isavuconazonium monosulfate of formula (I) comprising the following steps:
i) reacting isavuconazole (1) with N-methyl-N-(3-[((N-tertbutoxycarbonyl-N-methylamino) acetoxy)methyl]pyridin-2-yl) carbamic acid 1-chloroethyl ester (2), and potassium iodide (KI), thus obtaining BOC-isavuconazonium iodide (3);
ii) dissolving BOC-isavuconazonium iodide (3) in an organic solvent, to give an organic phase,
and
iii-PS) following the “persulfate pathway” by:
   a) dissolving a persulfate salt in water, to give an aqueous phase,
   b) mixing the organic phase of step ii) with the aqueous phase of sub-step a) to allow the ion-exchange reaction between BOC-isavuconazonium iodide (3) and the persulfate salt, thus resulting in the formation of bis-BOC-isavuconazonium sulfate (4) in the organic phase and iodide salt in the aqueous phase,
   c) separating the organic phase and reacting bis-BOC-isavuconazonium sulfate (4) with sulfuric acid to give isavuconazonium bis(sulfate) (5),
   d) reacting isavuconazonium bis(sulfate) (5) with an organic acid alkali salt soluble in methanol, to give isavuconazonium monosulfate (I),
or
iii-AG) following the “Ag pathway” by:
   α) providing HCl in an organic solvent,
   β) adding HCl of sub-step α) to the organic phase of step ii) at a temperature of −5 °C to 5 °C to allow the formation of isavuconazonium iodide hydrochloride (6),
   γ) dissolving isavuconazonium iodide hydrochloride (6) in a solvent comprising water, methanol, THF, o a mixture thereof, and adding one of the following reactives:
      Ag₂SO₄,
      Ag₂O or Ag₂CO₃, and K₂SO₄, or
      Ag₂O and Ag₂SO₄,
   to allow the formation of isavuconazonium monosulfate (I),
   δ) adding methanol to promote the precipitation of AgI and AgCl,
and
iv) adding isopropyl acetate, to promote the precipitation of isavuconazonium monosulfate (I), filtering and washing the latter.

The process of the invention has proven to obtain isavuconazonium monosulfate in high yield and purity, as well as in a convenient and expedite way.

Preferably, in step i), isavuconazole (1) and N-methyl-N-(3-[((N-tertbutoxycarbonyl-N-methylamino) acetoxy)methyl]pyridin-2-yl) carbamic acid 1-chloroethyl ester (2) are in a molar ratio of 1:1 to 1:5, more preferably, 1:1 to 1:3.

In preferred embodiments, isavuconazole (1) and N-methyl-N-(3-[((N-tertbutoxycarbonyl-N-methylamino) acetoxy)methyl]pyridin-2-yl) carbamic acid 1-chloroethyl ester (2) are in a molar ratio of 1:1.1 to 1:1.5.

The reaction in step i) also involves potassium iodide (KI); preferably, isavuconazole (1) and KI are in a molar ratio of 1:1 to 1:5, more preferably, 1:1 to 1:3.

In preferred embodiments, isavuconazole (1) and KI are in a molar ratio of 1:1.1 to 1:1.5. In most preferred embodiments, in step i), N-methyl-N-(3-[((N-tertbutoxycarbonyl-N-methylamino) acetoxy)methyl]pyridin-2-yl) carbamic acid 1-chloroethyl ester (2) and potassium iodide (KI) are in an equimolar quantity or in a substantial equimolar quantity. Preferably, the reaction in step i) is performed under inert atmosphere, e.g. using argon or nitrogen flow.

Preferably, this reaction is performed under stirring and heating, more preferably at a temperature of 40-60 °C, in a suitable organic solvent, such as acetonitrile, ethyl acetate, isopropyl acetate, methylene chloride, or a mixture thereof, more preferably for at least 6 hours.

In preferred embodiments, in step i), the reaction is performed under nitrogen flow, under stirring and heating at a temperature of 45-50 °C, and in acetonitrile, for 20-26 hours. At the end of the reaction, BOC-isavuconazonium iodide (3) is obtained and the solvent is preferably distilled off under vacuum.

In step ii), BOC-isavuconazonium iodide (3) is dissolved in an organic solvent, to give an organic phase.

Suitable organic solvents for this step are ethyl acetate, isopropyl acetate, methylene chloride, or a mixture thereof, preferably ethyl acetate. The resulting organic phase is washed with water, preferably at least twice. The organic phase is then separated and distilled under vacuum to obtain an oily residue (compound 3). The residue is preferably dissolved in dry ethyl acetate (600 ml) and directly used in the next step.

As reported above, step iii) can be performed following two different pathways, i.e. the “persulfate pathway” or “iii-PS” (as exemplary depicted in FIG. 2) and the “Ag pathway” in or “iii-AG” (as exemplary depicted in FIG. 3), depending on the reactive selected for converting BOC-isavuconazonium iodide (3) into isavuconazonium monosulfate (I).

In the “persulfate pathway”, step iii-a), a persulfate salt is dissolved in water, to give an aqueous phase.

In step iii-b), the organic phase of step ii) is mixed with the aqueous phase of step iii-a) to allow the reaction between BOC-isavuconazonium iodide (3) and the persulfate salt, thus resulting in the formation of bis-BOC-isavuconazonium sulfate (4) in the organic phase and iodide salt in the aqueous phase.

Preferably, BOC-isavuconazonium iodide (3) and the persulfate salt are in a molar ratio of 5:1 to 1:1, more preferably, 3:1 to 1.5:1.

Preferably, said persulfate salt is sodium persulfate, potassium persulfate, ammonium persulfate, or a mixture thereof, more preferably potassium persulfate.

In preferred embodiments, said persulfate salt is potassium persulfate, and BOC-isavuconazonium iodide (3) and potassium persulfate are in a molar ratio of 2:1 to 1.5:1.

Preferably, a half of the aqueous phase (i.e. persulfate solution) is added to the organic phase and the resulting mixture is stirred, preferably for 5-20 minutes. The obtained coloured water-phase is discarded and the remaining half of the aqueous phase (i.e. persulfate solution) is added to the organic phase. The mixture is stirred again for 5-20 minutes, then the resulting coloured water-phase is discarded and the organic phase is washed with water.

In step iii-c), the organic phase is separated and bis-BOC-isavuconazonium sulfate (4) is reacted with sulfuric acid to give isavuconazonium bis(sulfate) (5).

Preferably, the organic phase obtained in step iv) is distilled under vacuum and the resulting residue (i.e. bis-BOC-isavuconazonium sulfate (4)) is dissolved in dry ethyl acetate.

The obtained solution is cooled, preferably down to a temperature equal or below 0 °C, and a diluted solution of H₂SO₄ 96% in ethyl acetate is dropped therein, causing a solid to form and precipitate, said solid being isavuconazonium bis(sulfate) (5).

Alternatively, the obtained solution is added with cyclo-hexane or n-hexane, then stirred, so obtaining a suspension of bis-BOC-isavuconazonium sulfate (4). Said suspension is filtered off and dried under vacuum, thus achieving solid bis-BOC-isavuconazonium sulfate (4). The latter is then dissolved in an organic solvent, thus cooled, preferably down to a temperature equal or below 0 °C, and a diluted solution of H₂SO₄ 96% in ethyl acetate is dropped therein, causing a solid to form and precipitate, said solid being isavuconazonium bis(sulfate) (5).

The compound (5) is preferably washed twice with ethyl acetate. The wet crude compound (5) obtained is preferably suspended in ethyl acetate, stirred, filtered, washed twice with ethyl acetate, and dried under vacuum, thus obtaining the compound (5) in a dried state.

In step iii-d), isavuconazonium bis(sulfate) (5) is reacted with an organic acid alkali salt soluble in methanol, to give isavuconazonium monosulfate (I).

Suitable organic acid alkali salts soluble in methanol are sodium or potassium salts of a, linear or branched, aliphatic C2-C10 carboxylic acid or ester, or a mixture thereof; preferably, said organic acid alkali salts are a sodium salt of a, linear or branched, aliphatic C2-C10 carboxylic ester; more preferably said organic acid alkali salt is sodium 2-ethylhexanoate.

Preferably, isavuconazonium bis(sulfate) (5) and said alkali salt are in molar ratio of 1:2 to 1:10, more preferably 1:3 to 1:5.

Preferably, the compound (5) and the alkali salt are dissolved in methanol and stirred at room temperature, preferably 20-25 °C, for at least 60 minutes. More preferably, silica gel can be also added with the aim to increase purity, while its subsequent removal can be easily performed by filtering.

The reaction of step iii-d) results in an ion exchange where sodium sulphate precipitates out the solution and filtered off, whereas the filtrate is preferably concentrated under vacuum to give an oily residue containing ISV monosulfate (I).

Alternatively, in the “Ag pathway”, step iii-α), HCl is provided in an organic solvent. Suitable organic solvents for this step are ethyl acetate, isopropyl acetate, methylene chloride, or a mixture thereof, preferably ethyl acetate.

In step iii-β), HCl of step iii-α) is added to the organic phase of step ii) at a temperature of −5 °C to 5 °C to allow the formation of isavuconazonium iodide hydrochloride (6). Preferably, the solution is then let to rise at 20-25 °C and stirred for 6-48 h, preferably 20-30 h. The resulting suspension is filtered and the cake obtained is washed with the organic solvent of step iii-α). The crude solid isavuconazonium iodide hydrochloride (6) is dried under vacuum at room temperature.

In step iii-γ), isavuconazonium iodide hydrochloride (6) is dissolved in a solvent comprising water, methanol, THF, o a mixture thereof, then one of the following reactives is added:
Ag₂SO₄,
Ag₂O or Ag₂CO₃, and K₂SO₄, or
Ag₂O and Ag₂SO₄,
   to allow the formation of isavuconazonium monosulfate (I).

Preferably, the solvent is water, methanol, THF (i.e. tetrahydrofurane), water/methanol or water/THF.

Preferably, the selected reactive above is added in a molar ratio with isavuconazonium iodide hydrochloride (6) of 3:1 to 1:3, more preferably, 2:1 to 1:2. In preferred embodiments, the molar ratio is about 1:1.

Preferably, step iii-γ) is carried out at 20-25 °C for about 1-2 h.

In step iii-δ), methanol is added to promote the precipitation of AgI and AgCl, while isavuconazonium monosulfate (I) keeps dissolved.

AgI and AgCl are then filtered off, whereas the filtrate is preferably concentrated under vacuum to give an oily residue containing ISV monosulfate (I).

In alternative embodiments, once crude isavuconazonium iodide hydrochloride (6) is obtained in step β) of Ag pathway, and before performing the step γ), said crude isavuconazonium iodide hydrochloride (6) is subjected to purification via normal phase chromatography.

Preferably, crude isavuconazonium iodide hydrochloride (6) is dissolved in methanol before being injected into the chromatographic column, where the stationary phase comprises silica (preferably having particle size of 10-50 μm), and mobile phase comprises a mixture of acetone and methanol, preferably in a ratio 4:1.

This additional optional step of purification of crude isavuconazonium iodide hydrochloride (6) allows to achieve a purity of higher than 99.5%, starting from a purity of about 90% at the end of step β), so that the purified isavuconazonium iodide hydrochloride (6) in the subsequent step γ) of the process accordingly can give an even purer isavuconazonium monosulfate, independently of the reactive then selected.

This additional optional step has been performed with the aim of exploring the possibilities of further boosting the already optimal purity of isavuconazonium monosulfate of formula (I) obtained by the process of the invention, in case particular medical applications of the same would request an even higher purity.

These alternative embodiments demonstrate that the process of the invention can be successfully used also as a basis for boosting the final purity of isavuconazonium monosulfate of formula (I) at percentages definitely close to 100%.

In step iv), isavuconazonium monosulfate (I) either deriving from the “persulfate pathway” or the “Ag pathway”, is added with isopropyl acetate. The mixture is stirred for about 1 h, then filtered off under suction. The resulting cake is washed with ethyl acetate, and dried under vacuum, thus obtaining ISV monosulfate (I), as a yellow powder, in a high yield, preferably at least 90%.

In particular, the isavuconazonium monosulfate (I) obtainable by the process of the invention as described above, has a HPLC purity of ≥95%, and a sulfate content (IC) of 10.5-13%.

Considering that the theorical SO₄²⁻ content by Ionic Chromatography is 11.8%, it should be appreciated that the sulphate content of the isavuconazonium monosulfate (I) obtainable by the process of the invention is advantageously and suitably aligned with the theorical value.

This is particularly convenient as the process of the invention does not involve the use of ionic resin, column chromatography or freeze-drying step. Actually, as said above, chromatographic purifications and ion exchange chromatography for ion exchanges, as well as lyophilisation, are energetically demanding techniques and not suitable at all for high scale production.

Exclusively for sake of completeness and scientific purposes, the isavuconazonium monosulfate (I) obtained by the process of the invention and having a HPLC purity of ≥95%, has been subjected to a chromatographic purification via chromatographic column (stationary phase: silica—mobile phase: methylene chloride, or ethyl acetate and methanol, with increasing methanol gradient elution), thus obtaining an even purer isavuconazonium monosulfate (I). For example, starting from a isavuconazonium monosulfate (I) having a HPLC purity of about 97%, the additional step of purification via chromatographic column, has allowed to achieve a HPLC purity of ≥99%.

It should be also understood that all the combinations of preferred aspects of the process steps above described and the isavuconazonium monosulfate (I) obtainable by said process, are likewise preferred and deemed to be hereby disclosed.

It should be also understood that all the combinations of preferred aspects of the process of preparation of the invention, as above reported, are likewise preferred and deemed to be hereby disclosed also for the isavuconazonium monosulfate (I) obtainable by said process.

Below are working examples of the present invention provided for illustrative purposes.

### EXAMPLES

### Example 1

### Preparation of Isavuconazonium Monosulfate According to the Invention

A mixture of isavuconazole (Compound 1; 20 g, MW: 437.5; 45.7 mmol), N-methyl-N-(3-[((N-tertbutoxycarbonyl-N-methylamino)acetoxy)methyl]pyridin-2-yl) carbamic acid 1-chloroethyl ester (Compound 2; 26.2 g; MW: 413.9; 63.3 mmol), KI (10.5 g; MW: 166.0; 63.3 mmol) and acetonitrile (200 ml) was stirred at 45-50 °C under N₂ for 24 h (compound 1≤1% by HPLC). The solvent was distilled off under vacuum and ethyl acetate was added (600 ml). The mixture was stirred to obtain a complete dissolution, then the organic phase was washed twice with water (100 ml×2). The organic phase was separated and distilled under vacuum to obtain an oily residue (compound 3). The residue was dissolved in dry ethyl acetate (600 ml) and directly used for the next step. This solution is stable for almost 48 h, if stored at −15 °C.

The ethyl acetate solution of Compound 3 (equiv. 20 g of Compound 1; 45.7 mmol) obtained in the previous step was charged into a round bottom flask.

A solution of K₂S₂O₈ (6.8 g; MW: 270.32; 25.1 mmol) in water (200 ml) was prepared. A half of this persulfate solution was added to the previous organic phase and the mixture was stirred for about 15 minutes. The resulting coloured water-phase was discarded and the remaining half of the persulfate solution was added to the organic phase. The mixture was stirred for about 15 minutes, then the resulting coloured water-phase was separated and the organic phase was washed with water (100 ml×2). The organic phase was distilled under vacuum (T≤45 °C) and the obtained residue (Compound 4) was dissolved in dry ethyl acetate (600 ml). The solution was cooled to −10/0 °C and a diluted solution of H₂SO₄ 96% (17.2 g; 168.8 mmol) in ethyl acetate (100 ml) was dropped down into the solution in about 1.5 h maintaining the temperature at −10/0 °C.

A solid precipitated out the solution during the addition. The resulting suspension was allowed to rise at 20-25 °C and stirred for 25 h (Compound 4≤1.5% by HPLC), then filtered under suction and the resulting cake was washed twice with ethyl acetate (100 ml×2). The wet crude compound obtained was suspended in ethyl acetate (200 ml), stirred at 20-25 °C for 1 h, filtered, washed twice with ethyl acetate (100 ml×2) and dried under vacuum at 25 °C for about 18 h, thus obtaining the Compound 5, as a yellow hygroscopic powder (57.3 g). HPLC: ≥91.1%; Sulfate content (turbidimetry): 28.6%

Compound 5 (57 g; MW: 912.9; 62.4 mmol) and sodium 2-ethylhexanoate 97% (38.5 g; MW: 166.2; 224.6 mmol; 3.6 eq.) were dissolved in methanol (318 ml) and stirred at 20-25 °C for 120 minutes. The resulting salt which precipitated out the solution (Na₂SO₄) was filtered off and the clear filtrate was dropped down at 20-25 °C into another flask and distilled off under vacuum (T max 45 °C) to oily residue. The oil was dissolved in methanol (115 ml) and this solution was dropped down into another flask containing isopropyl acetate (1,710 ml) under stirring at 20-25 °C in about 1 h. The mixture was stirred for about 1 h, then filtered off under suction. The resulting cake was washed with ethyl acetate (200 ml), recovered and dried under vacuum at 20-30 °C for 24 h, thus obtaining ISV monosulfate of formula (I), as a yellow powder (28.7 g; overall yield on dried compound vs starting compound 1: 77%).

HPLC: ≥95.3%; Sulfate content (IC): 10.57%; Water content (KF titration): 1.54%

### Example 2

### Preparation of Isavuconazonium Monosulfate According to the Invention

A mixture of Isavuconazole (Compound 1; 20 g, MW: 437.5; 45.7 mmol), N-Methyl-N-(3-[((N-tertbutoxycarbonyl-N-methylamino)acetoxy)methyl]pyridin-2-yl) carbamic acid 1-chloroethyl ester (Compound 2; 26.2 g; MW: 413.9; 63.3 mmol), KI (10.5 g; MW: 166.0; 63.3 mmol) and acetonitrile (200 mL) was stirred at 45-50 °C under N₂ for 24 h (compound 1≤1% by HPLC). The solvent was distilled off under vacuum and ethyl acetate was added (600 mL). The mixture was stirred to obtain a complete dissolution, the dispersed salts was removed by filtration, then the organic phase was washed twice with water (100 mL×2). The organic phase was separated and directly used for the next step. This solution is stable for almost 48 h if stored at −15 °C.

The ethyl acetate solution of the resulting Compound 3 (equiv. 20 g of Compound 1; 45.7 mmol) obtained in the previous step was charged into a round bottom flask. A solution of K₂S₂O₈ (6.8 g; MW: 270.32; 25.1 mmol) in water (400 mL) was prepared. ¼ of this persulfate solution was added to the previous organic phase and the mixture was stirred for about 20 minutes. The resulting coloured water-phase was discarded and another ¼ of the persulfate solution was added to the organic phase, stirred for about 20 minutes and separated: this was made a total of 4 times. The resulted organic phase was washed with water (100 mL). The organic phase was distilled under vacuum (T≤45 °C) and the obtained residue was dissolved in dry ethyl acetate (160 mL). This solution was dropped down into a round bottom flask containing cyclohexane (1 L), stirred for about 30 minutes. The suspension was filtered under suction, the cake was washed with cyclohexane (40 mL). The collected solid was dissolved in dry ethyl acetate (160 mL). This solution was dropped down in a round bottom flask containing n-heptane (1 L), stirred for about 30 minutes and the suspension was filtered under suction. The cake was washed with n-heptane (40 mL). The collected yellow solid was dried under vacuum at room temperature for about 20 hours obtaining Compound 4 as a yellow powder (29.2 g).

HPLC: ≥92%.

### Compound 5:

In a round bottom flask, Compound 4 (29.2 g; MW: 1730; 16.6 mmol) was dissolved in ethyl acetate (600 mL). The solution was cooled down to −10/−5 °C and a diluted solution of H₂SO₄ 96% (14.7 g; 143.5 mmol) in ethyl acetate (100 mL) was dropped down into the solution in about 2 h maintaining the temperature at −10/−5 °C. A solid precipitated out the solution during the addition. The resulting suspension was allowed to rise at 20-25 °C and stirred for 25 h (Compound 4≤1% by HPLC), then filtered under suction and the cake was washed twice with ethyl acetate (100 mL×2). The wet crude compound so obtained was dried under vacuum at 25 °C for about 18 h, thus obtaining Compound 5 as a yellow hygroscopic powder (34.5 g).

HPLC: ≥90%; Sulfate content (turbidimetry): 23.3% (theoretical content 21.0%).

### Crude ISV Sulfate:

Compound 5 (34.5 g; MW: 912.9; 37.8 mmol) and sodium 2-ethylhexanoate 97% (16.1 g; MW: 166.2; 93.8 mmol; the amount of sodium 2-ethylhexanoate was calculated based on the % of sulfate into the starting material and then adding about 2.5 eq. of sodium 2-ethylhexanoate compared to this amount of sulfate) were dissolved in methanol (318 ml) and stirred at 20-25 °C for 60 minutes, after this period was added silica gel (3.5 g) and stirred at 20-25 °C for about 60 min. The resulting salt which precipitated out the solution (Na₂SO₄) and silica were filtered off and the clear filtrate was dropped down at 20-25 °C into another flask and distilled off under vacuum (T max 45 °C) to oily residue. The oil was dissolved in methanol (69 ml) and this solution was dropped down into another flask containing isopropyl acetate (1380 ml) under stirring at 20-25 °C in about 1 h. The mixture was stirred for about 1 h, then filtered under suction. The cake was washed with isopropyl acetate (200 ml), recovered and dried under vacuum at 20-30 °C for 24 h, thus obtaining ISV sulfate as a pale yellow powder (28.7 g; overall yield on dried compound vs starting Compound 1: 77%).

HPLC: 96.1%

### ISV Sulfate of Formula (I):

Crude ISV sulfate (28.7 g) was dissolved in methanol (143.5 mL) and dropped, in about 1 hour, in a round bottom flask containing isopropanol (1435 mL) and methyl t-butyl ether (1435 mL) under stirring. This mixture was stirred for about 2 hours, then filtered and the cake was washed twice with a solution 50:50 isopropanol:methyl t-butyl ether (143 mL×2), recovered and dried under vacuum at 20-30 °C for 20-24 h, thus obtaining ISV sulfate of formula (I) as a white powder (14.3 g; overall yield on dried compound vs starting Compound 1:38.4%)

HLPC >>96%; Sulfate content (turbidimetry): 18.3% (theoretical content 11.8%).

### Example 3

### Preparation of Isavuconazonium Monosulfate According to the Invention

A mixture of Isavuconazole (Compound 1; 20 g, MW: 437.5; 45.7 mmol), N-Methyl-N-(3-[((N-tertbutoxycarbonyl-N-methylamino)acetoxy)methyl]pyridin-2-yl) carbamic acid 1-chloroethyl ester (Compound 2; 26.2 g; MW: 413.9; 63.3 mmol), KI (10.5 g; MW: 166.0; 63.3 mmol) and acetonitrile (200 mL) was stirred at 45-50 °C under N₂ for 24 h (Compound 1≤1% by HPLC). The solvent was distilled off under vacuum and ethyl acetate was added (600 mL). The mixture was stirred to obtain a complete dissolution, the dispersed salts was removed by filtration, then the organic phase was washed twice with water (100 mL×2). The organic phase was separated and directly used for the next step. This solution is stable for almost 48 h, if stored at −15 °C.

The ethyl acetate solution of the resulting Compound 3 (equiv. 20 g of Compound 1; 45.7 mmol) obtained in the previous step was charged into a round bottom flask. A solution of K₂S₂O₈ (6.8 g; MW: 270.32; 25.1 mmol) in water (400 mL) was prepared. ¼ of this persulfate solution was added to the previous organic phase and the mixture was stirred for about 20 minutes. The resulting coloured water-phase was discarded and another ¼ of the persulfate solution was added to the organic phase, stirred for about 20 minutes and separated, this was made a total of 4 times. The resulted organic phase was washed with water (100 mL). The organic phase was distilled under vacuum (T≤45 °C) and the obtained residue was dissolved in dry ethyl acetate (160 mL). This solution was dropped down into a round bottom flask containing n-hexane (IL), stirred for about 30 minutes. The suspension was filtered under suction, the cake was washed with n-hexane (40 mL). The collected yellow solid was dried under vacuum at room temperature for about 20 hours, thus obtaining Compound 4 as a yellow powder (44.2 g).

HPLC: ≥90%.

### Compound 5:

In a round bottom flask, Compound 4 (44.2 g; MW: 1730; mmol: 25.5) was dissolved in ethyl acetate (600 mL). The solution was cooled to −10/−5 °C and a diluted solution of H₂SO₄ 96% (13.0 g; 127.7 mmol) in ethyl acetate (100 mL) was dropped down into the solution in about 2 h maintaining the temperature at −10/−5 °C. A solid precipitated out the solution during the addition. The resulting suspension was allowed to rise at 20-25 °C and stirred for 28 h (Compound 4≤3% by HPLC), then filtered under suction and the cake was washed twice with ethyl acetate (100 mL×2). The wet crude compound obtained was dried under vacuum at 25 °C for about 18 h, thus obtaining Compound 5 as a yellow hygroscopic powder (46.0 g).

HPLC: ≥92%; Sulfate content (turbidimetry): 20.52% (theoretical content 21.0%).

### Crude ISV Sulfate:

Compound 5 (46.0 g; MW: 912.9; 50.4 mmol) and sodium 2-ethylhexanoate 97% (21.8 g; MW: 166.2; 131.0 mmol; the amount of sodium 2-ethylhexanoate was calculated based on the % of sulfate into the starting material and adding about 2.5 eq. of sodium 2-ethylhexanoate compared to this amount of sulfate) were dissolved in methanol (318 ml) and stirred at 20-25 °C for 60 minutes, after this period was added silica gel (23 g) and stirred at 20-25 °C for about 60 min. The resulting salt which precipitated out the solution (Na₂SO₄) and silica were filtered off and the clear filtrate was dropped down at 20-25 °C into another flask and distilled off under vacuum (T max 45 °C) to oily residue. The oil was dissolved in methanol (92 ml) and this solution was dropped down into another flask containing isopropyl acetate (1840 ml) under stirring at 20-25 °C in about 1 h. The mixture was stirred for about 1 h, then filtered under suction. The cake was washed with isopropyl acetate (300 ml), recovered and dried under vacuum at 20-30 °C for 24 h, thus obtaining ISV sulfate as a pale yellow powder (20.0 g; overall yield on dried compound vs starting Compound 1: 54%).

HPLC: ≥96.6%

### ISV Sulfate of Formula (I):

Crude ISV sulfate (20.0 g) was dissolved in methanol (100 mL) and dropped, in about 1 hour, in a round bottom flask containing isopropanol (1000 mL) and methyl t-butyl ether (1000 mL) under stirring. This mixture was stirred for about 2 hours, then filtered and the cake was washed twice with a solution 50:50 isopropanol:methyl t-butyl ether (100 mL×2), recovered and dried under vacuum at 20-30 °C for 20-24 h, thus obtaining ISV sulfate of formula (I), as a white powder (12.9 g; overall yield on dried compound vs starting Compound 1:34.7%)

HLPC: >>96%; Sulfate content (turbidimetry): 14.8% (theoretical content 11.8%).

### Example 4

### Preparation of Isavuconazonium Monosulfate According to the Invention

### Compound 6 (Isavuconazonium Iodide Hydrochloride)

A mixture of Isavuconazole (Compound 1; 10 g, MW: 437.5; 22.85 mmol), N-Methyl-N-(3-[((N-tertbutoxycarbonyl-N-methylamino)acetoxy)methyl]pyridin-2-yl) carbamic acid 1-chloroethyl ester 14.2 g; MW: 413.9; 34.3 mmol; 1.5 eq.), KI (5.7 g; MW: 166.0; 34.3 mmol) and acetonitrile (100 ml) was stirred at 45-50 °C under N₂ for 24 h (Compound 1≤1% by HPLC). The solvent was distilled off under vacuum and ethyl acetate was added (300 ml). The mixture was stirred to obtain a complete dissolution, then the organic phase was washed twice with water (50 ml×2). The organic phase was separated and distilled under vacuum to obtain an oily residue. The residue was dissolved in dry ethyl acetate (300 ml) and directly used for the next step.

The ethyl acetate solution of Compound 3 (equiv. 10 g of Compound 1; 22.85 mmol) obtained in the previous step was cooled to −5/0 °C and HCl 1M in ethyl acetate (137.1 ml; 137.1 mmol; 6 eq.) was dropped down into the solution maintaining the temperature at −5/+5 °C. The solution was then allowed to rise at 20-25 °C and stirred for 24 h. The resulting suspension was filtered and the cake was washed twice with ethyl acetate (50 ml×2). The crude solid Compound 6 was dried under vacuum at room temperature for 24 h.

Yellow hygroscopic powder (25.1 g).

This intermediate is stored at −15 °C.

i) Isavuconazonium Sulfate of Formula (I) Obtained Via Ag₂SO₄

Compound 6 (5 g, 6.3 mmol) was dissolved in water (50 ml). Ag₂SO₄ (1.77 g, MW 311.8, 5.7 mmol) was added to the solution. The mixture was stirred at 20-25 °C for 0.5-1 h, then was diluted with methanol (25 ml). The solid which precipitated out the solution (AgI, AgCl) was filtered off and the filtrate was distilled under vacuum keeping the temperature below 40 °C to obtain an oily residue. Finally, the residue was diluted with methanol (about 10 ml) and the resulting solution was dropped into a large amount of isopropyl acetate (200 ml). The resulting suspension was stirred for 1 h at 20-25 °C, then filtered under suction and the cake was washed with isopropyl acetate, recovered and dried under vacuum at 20-30 °C for 5 hours.

Isavuconazonium sulfate of formula (I) was obtained as an off white powder (4.3 g; LOD 8.25%). [LOD means Loss on Drying, on a thermobalance, warm up to constant weight, thus indirectly giving volatile impurities, such as water or residual solvents]

HPLC: 96%; Sulfate content (Turbidimetry): 10.77%

ii) Isavuconazonium Sulfate of Formula (I) Obtained Via Ag₂O/K₂SO₄

Compound 6 (5 g; 6.3 mmol) was dissolved in water (50 ml). Ag₂O (1.47 g, 6.3 mmol) was added to the solution. The mixture was stirred at 20-25 °C for 2 h, then it was diluted with methanol (50 ml) and the solid which precipitated out the solution (AgI, AgCl) was filtered off. K₂SO₄ (1.1 g, 6.3 mmol) was added. The mixture was stirred for 0.5 h. The mixture was concentrated under vacuum, then diluted with 2-propanol (15 ml) and methanol (15 ml). The resulting solution was dropped down into another flask containing isopropyl acetate (300 ml). The resulting suspension was stirred for 2 h, then filtered and the cake was washed with isopropyl acetate. The cake was recovered and dried under vacuum at 20-30 °C for 24 h.

Isavuconazonium sulfate of formula (I) was obtained as a yellow powder (4.3 g; LOD 8.25%).

HPLC: 90%; Sulfate content (Turbidimetry): 7.4%

iii) Isavuconazonium Sulfate of Formula (I) Obtained Via Ag₂O/Ag₂SO₄

Compound 6 (5 g; 0.0057 mmol) was dissolved in water (50 ml). The water solution obtained was extracted with ethyl acetate (25 ml×5). The water phase containing the product was cooled at 0-5 °C. Ag₂SO₄ (1.42 g; 0.8 eq) and Ag₂O (1.06 g; 0.8 eq) were added and the resulting suspension was stirred at 0-5 °C for 1 h. Methanol was added (75 ml) and the resulting mixture was filtered through a glass filter. The filtrate solution was distilled under vacuum at 30 °C to obtain an oily residue, which was dissolved in methanol (10 ml). This methanolic solution was dropped down into another flask containing ethyl acetate (250 ml) at 10-15 °C. The mixture was stirred at 10-15 °C for 10 minutes. The resulting suspension was filtered off and the resulting cake was washed with ethyl acetate. The isolated solid was dried under vacuum at 20-25 °C for 16 h.

Isavuconazonium sulfate of formula (I) was obtained as an off white powder (2.15 g; Sulfate content (turbidimetry): 13.64%; HPLC: 95%).

### Example 5

### Preparation of Isavuconazonium Monosulfate According to the Invention

The crude solid Compound 6 was prepared as in Example 4, and then is stored at −15 °C. Before being subjected to the last step of the process of the invention to give isavuconazonium monosulfate, as per any one of options i) to iii), Compound 6 was previously purified via normal phase chromatography.

In details, a Reveleris® PREP purification system (i.e. a powerful, high-performance system that combines flash chromatography and preparative HPLC capabilities in a single instrument) was equipped with a column Biotage® Sfär Silica HC, while having set the following:

Column: Biotage ® Sfär Silica HC

| Silica | Particle | Diameter | Length | CV |
|---|---|---|---|---|
| (g) | size (μ) | (cm) | (cm) | (mL) |
| 350 | 20 | 6.5 | 25.5 | 530 |

### Settings of Reveleris Instrument:

Crude compound 35g
Cartridge: Biotage® Sfär Silica HC
Mobile phase: Acetone 80/Methanol 20-Isocratic
Flow: 80 mL/min
Temperature: room temperature (i.e. about 20 °C)
Injection: 35 g dissolved in 30 mL of methanol
Detection: UV
UV Threshold: 0.2 AU

For a run with a Biotage® column (350 g silica, 20μ), 6.3 L of solvents were consumed:
1.350 L of acetone/MeOH 80/20 for the initial equilibration
2.3 L of acetone/MeOH 80/20 for the elution
1.350 L of MeOH for the final washing of the column

With a single column, it was possible to perform 6 runs.

The following conditions were found to give the best results in terms of purification of Compound 6:

| | | | Crude | No of runs | | | | | Final | Final yield |
|---|---|---|---|---|---|---|---|---|---|---|
| Silica | Particle | Crude | purity | with the | Ambient | Wash | Elution | Time | purity of | of purified |
| (g) | size (μ) | (g) | (%) | same column | (L) | (L) | (L) | (min) | Compound 6 | Compound 6 |
| 350 | 20 | 35 | 90 | 6 | 1.350 | 1.350 | 2.3 | 80 | 99.8% | 50-55% |

What above shows that the additional optional step of purification of crude Compound 6 (purity of 90%) allows to achieve a purity of 99.8%, so that the purified Compound 6 in the subsequent last step of the process accordingly can give an even purer isavuconazonium monosulfate, independently of the reactives i) to iii) of step γ) then selected.

This additional optional step has been performed with the aim of exploring the possibilities of further boosting the already optimal purity of isavuconazonium monosulfate of formula (I) obtained by the process of the invention, in case particular medical applications of the same would request an even higher purity.

This example has demonstrated that the process of the invention can be successfully used also as a basis for boosting the final purity of isavuconazonium monosulfate of formula (I) at percentages definitely close to 100%.

### Comparative Example 1

The preparation pathway reported in CN106916152A has been repeated with the aim to demonstrate that it is not suitable to produce the desired sulfate salt in high yield and HPLC purity.

In brief, CN106916152A describes a process starting from compound 3 which is converted into compound 4 by reaction with CuSO₄. Compound 4 is then directly converted into ISV sulfate with sulfuric acid, as shown in FIG. 1. In this case, the iodine oxidizing species is the cation of the salt (Cu II to Cu I).

Moreover, the sulfate content by Ionic Chromatography declared by this patent application are 15.28% (example 3) and 15.25% (example 6), while the theoretical amount should be:
Theorical SO₄²⁻ 11.8%*
Theorical bis-HSO₄⁻ 21.0%
* [this percentage values are calculated on molecular weight basis, e.g. the MW of SO₄²⁻ (96.08) based on the overall MW of ISV monosulfate (814.83) gives 11.8% of sulphate content]

In repeating Example 3 and Example 6 of CN106916152A, it was observed that the process described therein allows to isolate ISV sulfate in a very low yield and at very low HPLC purity, mainly because of the deprotection condition, performed at high temperature in all the examples. Under these conditions, the hydrolysis of ISV sulfate occurs returning back to the starting isavuconazole, as the main impurity:
Experimental (CN106916152A)

ISV (5 g; 0.0114 mol), N-methyl-N-(3-[((N-tertbutoxycarbonyl-N-methylamino) acetoxy)methyl]pyridin-2-yl) carbamic acid 1-chloroethyl ester (6.65 g, 0.0160 mol), NaI (2.4 g) were charged into a round bottom flask and acetonitrile (25 ml) was added. The resulting suspension was stirred at 60 °C for 12 h. The suspension was filtered and washed with ethyl acetate (20 ml), than the filtrate was treated with water (50 ml). The mixture was stirred, then the water phase was discharged. The organic phase was washed once again with water (50 ml). The organic phase was distilled off obtaining a foamy solid. This solid was dissolved in ethyl acetate (45 ml), then water was added (45 ml). CuSO₄*5H₂O was added to the biphasic mixture and the reaction was stirred for 1 h at room temperature. The aqueous phase was separated while the organic phase was washed with water (25 ml) and finally with brine (25 ml). The organic phase was distilled off under vacuum to obtain an oily residue (11.3 g).

This compound was not purified by flash chromatography to better compare the purity of the title compound obtained with this procedure with Isavuconazonium sulfate obtained according to the process of the instant invention (i.e. encompassing no chromatographic purification). The oily residue was directly dissolved in ethyl acetate (37 ml) and water (19 ml). The solution was cooled to 0-5 °C and H₂SO₄ 2M solution in ethyl acetate (0.67 g of H₂SO₄ 96% diluted with 3.3 ml of ethyl acetate) was dropped down into the mixture. The reaction was stirred at 60 °C for 2 h, then cooled to 20-25 °C and diluted with water (56 ml). Ethyl acetate was added (188 ml) e the aqueous phase was separated. The aqueous phase was then washed with ethyl acetate (56 ml), DCM (56 ml) and finally with heptane (56 ml).

The aqueous phase was recovered and water was distilled off under high vacuum thus obtaining an off white solid, in a very low amount (<1 g) and purity (HPLC <<90%). To better compare this technique with the process of the present invention, the solid was dissolved MeOH (10 ml) and the methanolic solution was dropped into a large amount of ethyl acetate (200 ml). The suspension was stirred for 1 h, then filtered off at room temperature and washed with ethyl acetate. The solid obtained was significantly less pure (HPLC: 89%) and characterized by a higher level of sulfate content (33% by turbidimetry), as compared to the theoretical amount for the mono-sulfate derivative.

1. A process for the preparation of isavuconazonium monosulfate of formula (I) comprising the following steps:
i) reacting isavuconazole (1) with N-methyl-N-(3-[((N-tertbutoxycarbonyl-N-methylamino) acetoxy)methyl]pyridin-2-yl) carbamic acid 1-chloroethyl ester (2), and potassium iodide (KI), thus obtaining BOC-isavuconazonium iodide (3);
ii) dissolving BOC-isavuconazonium iodide (3) in an organic solvent, to obtain an organic phase,
and
iii-PS) following a “persulfate pathway” by:
a) dissolving a persulfate salt in water, to obtain an aqueous phase,
b) mixing the organic phase of step ii) with the aqueous phase of sub-step a) to allow the ion-exchange reaction between BOC-isavuconazonium iodide (3) and the persulfate salt, thus resulting in the formation of bis-BOC-isavuconazonium sulfate (4) in the organic phase and iodide salt in the aqueous phase,
c) separating the organic phase and reacting bis-BOC-isavuconazonium sulfate (4) with sulfuric acid to obtain isavuconazonium bis(sulfate) (5),
d) reacting isavuconazonium bis(sulfate) (5) with an organic acid alkali salt soluble in methanol, to obtain isavuconazonium monosulfate (I),
or
iii-AG) following an “Ag pathway” by:
α) providing HCl in an organic solvent,
β) adding HCl of sub-step α) to the organic phase of step ii) at a temperature of −5 °C to 5 °C to allow the formation of isavuconazonium iodide hydrochloride (6),
γ) dissolving isavuconazonium iodide hydrochloride (6) in a solvent comprising water, methanol, THF, o a mixture thereof, and adding one of the following reactives:
Ag₂SO₄,
Ag₂O or Ag₂CO₃, and K₂SO₄, or
Ag₂O and Ag₂SO₄,
to allow the formation of isavuconazonium monosulfate (I),
δ) adding methanol to promote the precipitation of AgI and AgCl,
and
iv) adding isopropyl acetate, to promote the precipitation of isavuconazonium monosulfate (I), filtering and washing said isavuconazonium monosulfate (I).

2. The process of claim 1, wherein, in step i), isavuconazole (1) and N-methyl-N-(3-[((N-tertbutoxycarbonyl-N-methylamino) acetoxy)methyl]pyridin-2-yl) carbamic acid 1-chloroethyl ester (2) are in a molar ratio of 1:1 to 1:5.

3. The process of claim 1, wherein, in step i), isavuconazole (1) and KI are in a molar ratio of 1:1 to 1:5, preferably 1:1 to 1:3, more preferably 1:1.1 to 1:1.5.

4. The process of claim 1, wherein, in step ii), the organic solvent is ethyl acetate, isopropyl acetate, methylene chloride, or a mixture thereof.

5. The process of claim 1, wherein, in step iii-b), BOC-isavuconazonium iodide (3) and the persulfate salt are in a molar ratio of 5:1 to 1:1.

6. The process of claim 1, wherein the persulfate salt is sodium persulfate, potassium persulfate, ammonium persulfate, or a mixture thereof.

7. The process of claim 6, wherein the persulfate salt is potassium persulfate, and BOC-isavuconazonium iodide (3) and potassium persulfate are in a molar ratio of 2:1 to 1.5:1.

8. The process of claim 1, wherein, in step iii-d), the organic acid alkali salt soluble in methanol is a sodium or potassium salt of a, linear or branched, aliphatic C2-C10 carboxylic acid or ester, or a mixture thereof.

9. The process of claim 8, wherein the organic acid alkali salt soluble in methanol is a sodium salt of a, linear or branched, aliphatic C₂-C₁₀ carboxylic ester.

10. The process of claim 1, wherein, in step iii-d), isavuconazonium bis(sulfate) (5) and the organic acid alkali salt are in molar ratio of 1:2 to 1:10.

11. The process of claim 1, wherein, in step iii-α), the organic solvent is ethyl acetate, isopropyl acetate, methylene chloride, or a mixture thereof.

12. The process of claim 1, wherein, in step iii-β), HCl of step iii-α) is added to the organic phase of step ii) at a temperature of −5 °C to 5 °C, to allow the formation of isavuconazonium iodide hydrochloride (6), then let to rise at 20-25 °C and stirred for 6-48 h.

13. The process of claim 1, wherein, in step iii-γ), the reactive is added in a molar ratio with isavuconazonium iodide hydrochloride (6) of 3:1 to 1:3.

14. The process of claim 1, wherein, step iii-γ) is carried out at 20-25 °C for about 1-2 h.

15. Isavuconazonium monosulfate (I) obtained by the process of claim 1, having a HPLC purity of ≥95%, and a sulfate content (IC) of 10.5-13%.

16. The process of claim 1, wherein isavuconazole (1) and N-methyl-N-(3-[((N-tertbutoxycarbonyl-N-methylamino)acetoxy)methyl]pyridin-2-yl) carbamic acid 1-chloroethyl ester (2) are in a molar ratio of 1:1 to 1:3.

17. The process of claim 1, wherein isavuconazole (1) and N-methyl-N-(3-[((N-tertbutoxycarbonyl-N-methylamino)acetoxy)methyl]pyridin-2-yl) carbamic acid 1-chloroethyl ester (2) are in a molar ratio of 1:1.1 to 1:1.5.

18. The process of claim 1, wherein, in step i), isavuconazole (1) and KI are in a molar ratio of 1:1 to 1:3.

19. The process of claim 1, wherein, in step i), isavuconazole (1) and KI are in a molar ratio of 1:1.1 to 1:1.5.

20. The process of claim 1, wherein, in step iii-b), BOC-isavuconazonium iodide (3) and the persulfate salt are in a molar ratio of 3:1 to 1.5:1.
